(19) United States
(12) Patent Application Publication
(10) Pub. No.: US 2025/0001037 A1
(43) Pub. Date: Jan. 2, 2025

(54) Sterilization and Disinfection Lamp, Combined Lamp and Lamp

(71) Applicants: CRAWFORD GLOBAL LIMITED, Hong Kong (CN); ETI SOLID STATE LIGHTING INC, Wheeling, IL (US)

(72) Inventors: Donglei WANG, Zhuhai, Guangdong (CN); Bingbing ZHOU, Zhuhai, Guangdong (CN); Cong ZHOU, Zhuhai, Guangdong (CN); Wuzhi HU, Zhuhai, Guangdong (CN); Xiaoyun PENG, Zhuhai, Guangdong (CN); Jibin CHENG, Zhuhai, Guangdong (CN)

(21) Appl. No.: 18/701,202

(22) PCT Filed: Oct. 12, 2021
(86) PCT No.: PCT/CN2021/123350

§ 371 Date: Apr. 12, 2024

(51) Int. Cl. A61L 9/22; A61L 9/18; F21V 29/77; H05H 1/24

(52) U.S. Cl. CPC A61L 9/22; A61L 9/18; F21V 29/773; H05H 1/24; A61L 2209/12; A61L 2209/134; A61L 2209/14; A61L 2209/15; H05H 2245/10; H05H 2245/36

(57) ABSTRACT

The disclosure provides a sterilization and disinfection lamp, a combined lamp and a lamp. The sterilization and disinfection lamp comprises a housing, wherein the housing is provided with a sterilization cavity, and an air inlet and an air outlet, which communicate with the sterilization cavity; a light-emitting assembly, which is mounted on an outer surface of the housing; and a plasma generator, at least part of which is accommodated in the sterilization cavity and generates positive ions and negative ions, wherein at least some positive ions and negative ions are combined in the sterilization cavity to sterilize and disinfect air flowing into the sterilization cavity through the air inlet, By means of the sterilization and disinfection lamp, the combined lamp and the lamp provided by the disclosure, the problems of air sterilization devices in the prior art affecting human health and having a single function may be solved.

### CROSS-REFERENCE TO RELATED APPLICATION

The disclosure is a National Stage Filing of the PCT International Application No. PCT/CN2021/123350 filed on Oct. 12, 2021, which is hereby incorporated by reference in its entirety.

### TECHNICAL FIELD

The disclosure relates to the technical field of sterilization equipment, specifically to a sterilization and disinfection lamp, a combined lamp and a lamp.

### BACKGROUND

There are a large number of bacteria and viruses in the air. During the process of breathing of people, bacteria and viruses enter the human body, which increases the burden on the immune system of the human body, accelerates the power consumption of the human body, and reduces the vitality of the human body.

In order to sterilize bacteria and viruses in the air, the current air sterilization devices mainly use the means of disinfectants, ozone and ultraviolet sterilization. However, disinfectants may generate an odor that stimulates the body, which may easily cause discomfort to the human body; in the process of using ozone to sterilize the air, people often need to stay away from sterilization places to reduce the impact on the human body, and a ventilation operation is required, which increases the activity of the human body and leads to low sterilization efficiency; and in the process of using ultraviolet sterilization to sterilize the air, it is easy to cause harm to the human body. Furthermore, the above-mentioned means of sterilization only have a sterilization function, so the function is single.

As may be seen from the above, the current air sterilization devices have the problems of affecting human health and having a single function.

### SUMMARY

The main purpose of the disclosure is to provide a sterilization and disinfection lamp, a combined lamp and a lamp to solve the problems of air sterilization devices in the prior art affecting human health and having a single function.

In order to achieve the above purpose, according to one aspect of the disclosure, a sterilization and disinfection lamp is provided. The sterilization and disinfection lamp includes a housing, wherein the housing is provided with a sterilization cavity, and an air inlet and an air outlet, which communicate with the sterilization cavity, and the air inlet is arranged on a bottom surface of the housing; a light-emitting assembly, which is mounted on an outer surface of the housing; and a plasma generator, at least part of which is accommodated in the sterilization cavity and generates positive ions and negative ions, wherein at least some positive ions and negative ions are combined in the sterilization cavity to sterilize and disinfect air flowing into the sterilization cavity through the air inlet, and at least some other positive ions and negative ions flow into the outside environment through the air outlet and are combined, so as to sterilize and disinfect the air of the outside environment.

In some embodiments, the air inlet or the air outlet is located at a center of the bottom surface of the housing.

In some embodiments, the air inlet is arranged on the bottom surface of the housing, one or a plurality of air inlets are provided, the plurality of air inlets are arranged in a ring structure in cooperation, and adjacent air inlets are arranged at an interval.

In some embodiments, the air inlet is arranged on a side surface of the housing, one or a plurality of air inlets are provided, and when a plurality of air inlets are provided, the air inlets are arranged at intervals along a circumferential direction of the housing; and/or the air inlets are arranged at intervals along a height direction of the housing.

In some embodiments, the air outlet is arranged on the bottom surface of the housing, one or a plurality of air outlets are provided, and when a plurality of air outlets are provided, the plurality of air outlets are arranged in a ring structure in cooperation, and adjacent air outlets are arranged at an interval.

In some embodiments, the air outlet is arranged on the side surface of the housing, one or a plurality of air outlets are provided, and when a plurality of air outlets are provided, the air outlets are arranged at intervals along the circumferential direction of the housing; and/or the air outlets are arranged at intervals along the height direction of the housing.

In some embodiments, the light-emitting assembly is arranged on the bottom surface of the housing; and/or the light-emitting assembly is arranged in a circumferential direction of the air inlet and/or the air outlet on the bottom surface of the housing.

In some embodiments, the sterilization and disinfection lamp further includes a fan, the fan is a centrifugal fan, the air inlet is arranged on the bottom surface of the housing, the air outlet is arranged on the side surface of the housing and/or the air outlet is arranged on the bottom surface of the housing, the fan is used for blowing the positive ions and the negative ions into the outside environment from the air outlet, the fan is directly opposite to the air inlet so that the air from the air inlet uniformly flows to the air outlet through the fan, and an emission head assembly of the plasma generator is arranged close to the air outlet relative to the fan; or the sterilization and disinfection lamp further includes a fan, the fan is a reverse centrifugal fan, the air outlet is arranged on the bottom surface of the housing, the air inlet is arranged on the side surface of the housing and/or the air inlet is arranged on the bottom surface of the housing, the fan is used for blowing the positive ions and the negative ions into the outside environment from the air outlet, and an emission head assembly of the plasma generator is arranged close to the air outlet relative to the fan.

In some embodiments, a plurality of air guide ribs are disposed on a surface of the bottom surface of the housing towards the interior of the housing, the plurality of air guide ribs are arranged in a ring structure, two adjacent air guide ribs are arranged at an interval, an air guide duct is formed between two adjacent air guide ribs, the air passing area of the air guide duct gradually increases along an air outlet direction, one end of the air guide duct communicates with the air inlet, and the other end of the air guide duct communicates with the air outlet; and/or the air guide rib is an arc-shaped section and the air guide rib is provided with a first radian, and the first radian is greater than 0° and less than 90°.

In some embodiments, the plasma generator includes a plasma flyback transformer, which is mounted on the housing; and an emission head assembly, which is arranged on a cavity wall of the sterilization cavity, wherein the plasma flyback transformer is connected to the emission head assembly, at least an emission head of the emission head assembly is arranged inside the air guide duct, and at least one emission head assembly is arranged inside at least one air guide duct.

In some embodiments, a plurality of emission head assemblies are provided, the plurality of emission head assemblies are arranged at intervals along a circumferential direction of the cavity wall of the sterilization cavity, and the emission head of each emission head assembly is exposed in the sterilization cavity; and/or the distance between two adjacent emission head assemblies in the plurality of emission head assemblies is the same; and/or the plasma generator includes one of a high-energy plasma generator and a low-energy plasma generator.

In some embodiments, the emission head assembly includes a positive ion emission head and a negative ion emission head, the positive ion emission head is parallel to the negative ion emission head, and an extension direction of the positive ion emission head and the negative ion emission head is perpendicular or horizontal to an air flow direction in the sterilization cavity.

In some embodiments, the air outlet is provided with an air outlet grid, an air guide surface of the air outlet grid is obliquely arranged relative to a horizontal plane, and the air guide surface obliquely extends downward along an air outlet direction.

In some embodiments, the sterilization and disinfection lamp further includes a filter sieve, the filter sieve is detachably arranged, and the filter sieve is arranged at the air inlet to filter the air entering the inside of the housing; and/or the bottom surface of the housing is provided with a convex rib extending towards an air inlet direction, the convex rib encloses an air inlet channel, an inlet of the air inlet channel is the air inlet, the filter sieve is arranged in the air inlet channel, and the filter sieve and the air inlet are arranged at an interval.

In some embodiments, the sterilization and disinfection lamp further includes a junction box arranged on an outer side of the housing, the junction box is arranged on a surface of the housing away from the air inlet, a power supply, a circuit module and a switch are arranged inside the junction box, the power supply is electrically connected to the circuit module through the switch, and the circuit module is electrically connected to the fan and/or the plasma generator and/or the light-emitting assembly of the sterilization and disinfection lamp.

In some embodiments, the sterilization and disinfection lamp further includes an indication lamp, the indication lamp is electrically connected to the circuit module, and at least part of the indication lamp is located on the bottom surface of the housing and/or the side surface of the housing.

In some embodiments, the sterilization and disinfection lamp further includes a radiator, and the radiator is padded between the light-emitting assembly and the bottom surface of the housing; and/or the radiator is arranged in a cylinder structure, and the air inlet channel of the sterilization and disinfection lamp passes through the radiator.

In some embodiments, the sterilization and disinfection lamp further includes a fan, and the gas temperature inside the housing may be less than or equal to 50° by arranging the radiator and adjusting the air velocity and air volume of the fan.

In some embodiments, the light-emitting assembly includes a lamp panel and a light-pervious cover, and the light-pervious cover covers the outside of the lamp panel.

In some embodiments, the sterilization and disinfection lamp further includes a lampshade, the lampshade is a horn structure, the lampshade is provided with a first opening and a second opening, the diameter of the second opening is greater than the diameter of the first opening, the second opening is located below the first opening, the light-emitting assembly is located in the lampshade, and the air inlet of the sterilization and disinfection lamp faces the second opening.

In some embodiments, the sterilization and disinfection lamp further includes a connecting structure, the connecting structure includes at least one of a hanging hook, a suspension chain and a clamp hook, and the connecting structure is arranged on a top surface of the housing.

In some embodiments, a mounting head of the housing is provided with a first connecting structure, the sterilization and disinfection lamp further includes a mounting base, the mounting base is provided with a second connecting structure in cooperation with the first connecting structure, and the first connecting structure and the second connecting structure may be connected in place to supply power to the sterilization and disinfection lamp.

In some embodiments, an outer surface of the mounting head is provided with a first mounting thread, the mounting base is provided with a mounting groove, the mounting groove is provided with a second mounting thread in cooperation with the first mounting thread, the mounting base supplies power to the sterilization and disinfection lamp after the first mounting thread and the second mounting thread are connected in place, the first mounting thread serves as the first connecting structure, and the second mounting thread serves as the second connecting structure; or an outer surface of the mounting head is provided with a first clamping structure, the mounting base is provided with a second clamping structure in cooperation with the first clamping structure, the mounting base supplies power to the sterilization and disinfection lamp after the first clamping structure and the second clamping structure are clamped in place, the first clamping structure serves as the first connecting structure, and the second clamping structure serves as the second connecting structure; or the first connecting structure is fixedly connected to the second connecting structure; or the first connection structure is connected to the second connection structure line.

In some embodiments, the sterilization and disinfection lamp further includes a support post, one end of the support post being connected to the mounting base, the support post is a hollow structure, and at least part of wires being accommodated in the support post; and a chassis, the other end of the support post being connected to the chassis, wherein the chassis may be mounted on a wall to enable the sterilization and disinfection lamp hang on the wall, or the chassis may be placed on a table top to enable the sterilization and disinfection lamp to stand upright.

In some embodiments, the sterilization and disinfection lamp further includes a cover body, the cover body is connected to the mounting base or the cover body is connected to the support post, and the light-emitting assembly is located in the cover body; and/or the sterilization and disinfection lamp further includes a support rod, and the support rod is connected to the chassis and the cover body.

According to another aspect of the disclosure, a combined lamp is provided. The combined lamp includes the above sterilization and disinfection lamp and an illuminating lamp. The illuminating lamp is connected to the sterilization and disinfection lamp, the illuminating lamp includes a lamp housing and a light source arranged inside the lamp housing, and the lamp housing is detachably connected to the housing of the sterilization and disinfection lamp.

In some embodiments, the combined lamp includes at least one light-emitting group, and each light-emitting group is composed of a sterilization and disinfection lamp unit and an illuminating lamp unit, wherein each sterilization and disinfection lamp unit includes one or a plurality of sterilization and disinfection lamps, each illuminating lamp unit includes one or a plurality of illuminating lamps, and the light-emitting group includes one sterilization and disinfection lamp unit and one illuminating lamp unit; or two sterilization and disinfection lamp units and one illuminating lamp unit, wherein the two sterilization and disinfection lamp units are respectively arranged at both ends of the illuminating lamp unit; or one sterilization and disinfection lamp unit and two illuminating lamp units, wherein the two illuminating lamp units are respectively arranged at both ends of the sterilization and disinfection lamp unit.

In some embodiments, the combined lamp further includes a connector and a connecting post, one end of the connecting post is connected to the top surface of the housing of the sterilization and disinfection lamp, the other end of the connecting post is connected to the connector, the illuminating lamp is connected to the sterilization and disinfection lamp or the illuminating lamp is connected to the connecting post or the illuminating lamp is connected to the connector, the connector may be mounted on a wall to enable the sterilization and disinfection lamp hang on the wall, or the connector may be placed on a table top to enable the sterilization and disinfection lamp to stand upright.

In some embodiments, the combined lamp further includes a plurality of connecting rods, one end of the connecting rod is connected to the illuminating lamp, and the other end of the connecting rod is connected to the connector; and/or the combined lamp further includes a lamp ring, the lamp ring is arranged along a circumferential direction of the sterilization and disinfection lamp, the lamp ring is provided with a plurality of lamp mounting positions for mounting the illuminating lamp, and the lamp ring is connected to the connecting rod or the lamp ring is connected the housing or the lamp ring is connected the connecting post.

According to another aspect of the disclosure, a lamp is provided. The lamp includes the above housing, the fan of the above sterilization and disinfection lamp and the above plasma generator, wherein the housing, the fan and the plasma generator cooperate to form a sterilization module; and an illuminating lamp, wherein the illuminating lamp is connected to the sterilization module, the illuminating lamp includes a lamp housing and a light source arranged inside the lamp housing, and the lamp housing is detachably connected to the housing.

In some embodiments, the lamp includes at least one light-emitting group, and each light-emitting group is composed of a sterilization module unit and an illuminating lamp unit, wherein each sterilization module unit includes one or a plurality of sterilization modules, each illuminating lamp unit includes one or a plurality of illuminating lamps, and the light-emitting group includes one sterilization module unit and one illuminating lamp unit; or two sterilization module units and one illuminating lamp unit, wherein the two sterilization module units are respectively arranged at both ends of the illuminating lamp unit; or one sterilization module unit and two illuminating lamp units, wherein the two illuminating lamp units are respectively arranged at both ends of the sterilization module unit.

In some embodiments, the lamp further includes a connector and a connecting post, one end of the connecting post is connected to the top surface of the housing of the sterilization module, the other end of the connecting post is connected to the connector, the illuminating lamp is connected to the sterilization module or the illuminating lamp is connected to the connecting post or the illuminating lamp is connected to the connector, the connector may be mounted on a wall to enable the sterilization module hang on the wall, or the connector may be placed on a table top to enable the sterilization module to stand upright.

In some embodiments, the lamp further includes a plurality of connecting rods, one end of the connecting rod is connected to the illuminating lamp, and the other end of the connecting rod is connected to the connector; and/or the lamp further includes a lamp ring, the lamp ring is arranged along a circumferential direction of the sterilization module, the lamp ring is provided with a plurality of lamp mounting positions for mounting the illuminating lamp, and the lamp ring is connected to the connecting rod or the lamp ring is connected to the housing or the lamp ring is connected to the connecting post.

In some embodiments, the illuminating lamp is provided with a first connecting part, the sterilization module is provided with a second connecting part, and the first connecting part and the second connecting part are independently mounted on a wall or a table top; and/or the first connecting part is connected to the second connecting part, and at least one of the first connecting part and the second connecting part is mounted on a wall or a table top; and/or the illuminating lamp includes one of a ceiling lamp, a hanging lamp, a floor lamp, a table lamp, a wall lamp, a recessed lamp, a projection lamp and a track lamp.

By applying the technical solutions of the disclosure, the fan is mounted inside the housing, air enters the inside of the housing from the air inlet on the housing through the fan, the air entering the inside of the housing passes through the plasma emitted by the plasma generator, the plasma is provided with positive ions and negative ions, the positive ions and the negative ions sterilize the air inside the housing, and under the action of airflow, some positive ions and negative ions diffuse into the air outside the housing through the air outlet following the air, so as to sterilize the air outside the housing.

An air outlet surface is arranged along a circumferential direction of an air inlet surface, so that the air from the air inlet uniformly diffuses to the outside of the housing through the air outlet under the action of the fan, thus increasing the sterilization efficiency.

The use of the plasma sterilization technology may effectively achieve man-machine symbiosis without affecting the human body. The housing is provided with a lamp panel mounting part, a lamp panel is mounted on the housing to form a combined lamp, and the combined lamp is provided with an illumination function and a sterilization function.

### BRIEF DESCRIPTION OF THE DRAWINGS

The accompanying drawings of the specification, constituting a part of the disclosure, are used for providing further understanding for the disclosure. Exemplary embodiments of the disclosure and descriptions thereof are used for explaining the disclosure, and do not constitute any inappropriate limitation to the disclosure. In the accompanying drawings:

FIG. 1 shows an exploded view of a sterilization and disinfection lamp in an embodiment 1 of the disclosure;

FIG. 2 shows a schematic view of a three-dimensional structure of the sterilization and disinfection lamp in the embodiment 1 of the disclosure;

FIG. 3 shows a bottom view of the sterilization and disinfection lamp in the embodiment 1 of the disclosure;

FIG. 4 shows a cross-sectional view of the sterilization and disinfection lamp in the embodiment 1 of the disclosure;

FIG. 5 shows a schematic view of a mounting structure of the sterilization and disinfection lamp in the embodiment 1 of the disclosure;

FIG. 6 shows an enlarged view of a part A in FIG. 5;

FIG. 7 shows an exploded view of a sterilization and disinfection lamp in an embodiment 3 of the disclosure;

FIG. 8 shows a cross-sectional view of the sterilization and disinfection lamp in the embodiment 3 of the disclosure;

FIG. 9 shows an exploded view of a sterilization and disinfection lamp in an embodiment 4 of the disclosure;

FIG. 10 shows a cross-sectional view of the sterilization and disinfection lamp in the embodiment 4 of the disclosure;

FIG. 11 shows a schematic view of a three-dimensional structure of a sterilization and disinfection lamp in an embodiment 5 of the disclosure;

FIG. 12 shows a schematic view of a three-dimensional structure of a sterilization and disinfection lamp in an embodiment 6 of the disclosure;

FIG. 13 shows a schematic view of a three-dimensional structure of the sterilization and disinfection lamp in the embodiment 6 of the disclosure;

FIG. 14 shows a schematic view of a three-dimensional structure of the sterilization and disinfection lamp in the embodiment 6 of the disclosure;

FIG. 15 shows a schematic view of a three-dimensional structure of the sterilization and disinfection lamp in the embodiment 6 of the disclosure;

FIG. 16 shows an exploded view of a sterilization and disinfection lamp in an embodiment 7 of the disclosure;

FIG. 17 shows a front view of the sterilization and disinfection lamp in the embodiment 7 of the disclosure;

FIG. 18 shows a cross-sectional view of the sterilization and disinfection lamp in the embodiment 7 of the disclosure;

FIG. 19 shows an exploded view of a sterilization and disinfection lamp in an embodiment 8 of the disclosure;

FIG. 20 shows a cross-sectional view of the sterilization and disinfection lamp in the embodiment 8 of the disclosure;

FIG. 21 shows an exploded view of a combined lamp in an embodiment 9 of the disclosure;

FIG. 22 shows a schematic view of a three-dimensional structure of the combined lamp in the embodiment 9 of the disclosure;

FIG. 23 shows a schematic view of a three-dimensional structure of the combined lamp in the embodiment 9 of the disclosure;

FIG. 24 shows a schematic view of a three-dimensional structure of the combined lamp in the embodiment 9 of the disclosure;

FIG. 25 shows an exploded view of the combined lamp in the embodiment 9 of the disclosure;

FIG. 26 shows a cross-sectional view of the combined lamp in the embodiment 9 of the disclosure;

FIG. 27 shows a schematic view of a three-dimensional structure of a combined lamp in an embodiment 10 of the disclosure; and

FIG. 28 shows a schematic view of another three-dimensional structure of the combined lamp in the embodiment 10 of the disclosure.

The above accompanying drawings have the following reference numerals:
100. housing; 1001. mounting head; 101. bottom surface of housing; 1011. air inlet; 102. side surface of housing; 1021. air outlet; 1022. air outlet grid; 103. lamp panel mounting part; 104. convex rib; 105. chassis; 106. support post; 107. cover body; 108. support rod; 201. plasma flyback transformer; 202. emission head assembly; 300. fan; 400. indication lamp; 401. mounting post; 500. lamp panel; 600. light-pervious cover; 700. filter sieve; 800. torsion spring assembly; 900. spring assembly; 1100. junction box; 1200. power supply; 1300. switch; 1400. illuminating lamp; 1401. air vent; 1402. light source; 1403. lamp housing; 1500. radiator; 1600. lampshade; 1700. lamp ring; 1800. connector; 1901. connecting post; 1902. connecting rod.

### DETAILED DESCRIPTION OF THE EMBODIMENTS

It should be noted that in the case of no conflict, the embodiments in the disclosure and the features in the embodiments may be combined with each other. The disclosure will be described in detail below with reference to the accompanying drawings in conjunction with the embodiments.

It should be noted that unless otherwise specified, all technical and scientific terms used in the disclosure have the same meanings as commonly understood by those skilled in the art of the disclosure.

In the disclosure, unless otherwise specified, the used directional words such as “upper, lower, top and bottom” are usually related to the directions shown in the accompanying drawings, or related to the upright, vertical or gravity directions of components. Similarly, for the convenience of understanding and description, “inside and outside” refer to the inside and outside relative to the contour of each component, but the above directional words are not intended to limit the disclosure.

In order to solve the problems of air sterilization devices in the prior art affecting human health and having a single function, the disclosure provides a sterilization and disinfection lamp, a combined lamp and a lamp. The sterilization and disinfection lamp, the combined lamp and the lamp may all be applied in household and industrial scenarios for sterilization and illumination.

### Embodiment 1

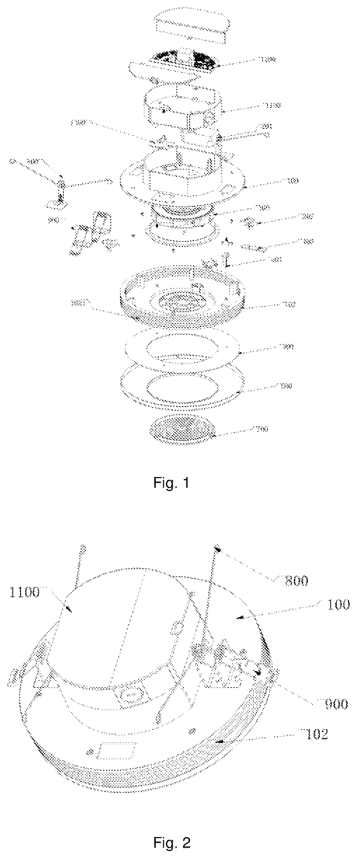

As shown in FIG. 1 and FIG. 6, the sterilization and disinfection lamp includes a housing 100, a light-emitting assembly and a plasma generator. The housing 100 is provided with a sterilization cavity, and an air inlet 1011 and an air outlet 1021, which communicate with the sterilization cavity, and the air inlet 1011 is arranged on a bottom surface 101 of the housing. The light-emitting assembly is mounted on an outer surface of the housing 100. At least part of the plasma generator is accommodated in the sterilization cavity and generates positive ions and negative ions, at least some positive ions and negative ions are combined in the sterilization cavity to sterilize and disinfect air flowing into the sterilization cavity through the air inlet, and at least some other positive ions and negative ions flow into the outside environment through the air outlet and are combined, so as to sterilize and disinfect the air of the outside environment.

Specifically, the air inside the housing 100 is in contact with the plasma emitted by the plasma generator, the plasma is provided with positive ions and negative ions, the positive ions and the negative ions sterilize the air inside the housing 100, and under the action of airflow, some positive ions and negative ions diffuse into the air outside the housing 100 through the air outlet 1021 following the air, so as to sterilize the air outside the housing 100.

The release of energy is generated at the moment when the positive ions and negative ions generated by the plasma generator are neutralized in the air, resulting in structure change or energy conversion of surrounding bacteria, thereby causing bacterial death and achieving a sterilization function.

The use of the plasma sterilization technology may effectively achieve man-machine symbiosis without affecting the human body. The light-emitting assembly may emit light, so that the light-emitting assembly is provided with both illumination and sterilization functions.

It should be noted that the plasma generator simultaneously generates spike pulse discharge through dual-electrode high voltage and ionizes air to form high-concentration plasma. At the same time, an emission head assembly 202 of the plasma generator releases a large number of high-energy electrons under the action of pulsed high voltage, and gas molecules collide with the high-energy electrons, resulting in the enrichment of ions, electrons, excited atoms, molecules and some active oxidizing substances such as OH in space. Strongly oxidizing substances are not easily obtained in general chemical reactions, but may be continuously produced in pulse discharge plasma channels. High-energy charged particles, active ingredients and the like may diffuse in space to collide and bind with bacteria, pathogens and the like, so as to ultimately lead to the inactivation and removal of bacteria and pathogens by disrupting the protein structures of cells. Furthermore, hydroxyl radicals and the like may disrupt the molecular structures of harmful gases to effectively remove odors. High-concentration negative air ions may degrade suspended particulate matters in the air and pollen pollution.

As shown in FIG. 1 to FIG. 6, the air inlet 1011 is located at a center of the bottom surface 101 of the housing, the bottom surface 101 of the housing is an air inlet surface, and the air inlet surface is provided with the air inlet 1011.

In some embodiments, the air inlet 1011 is not limited to be arranged at the center position of the bottom surface 101 of the housing, and the air inlet 1011 may also be offset relative to the center position of the bottom surface 101 of the housing, as long as the air may enter from the bottom surface 101 of the housing, that is, the air may enter the inside of the housing 100 from the air inlet surface.

In some embodiments, if one air inlet 1011 is provided, at this time, the air inlet 1011 may be located at the center position of the air inlet surface, so that after the air enters the inside of the housing 100, the air is uniformly distributed and discharged through the air outlet 1021. Furthermore, by arranging the air inlet 1011 at the center position, positive ions and negative ions may uniformly diffuse to the outside of the housing 100.

Of course, a plurality of air inlets 1011 may also be provided, and the plurality of air inlets 1011 are arranged along a circumferential direction of the center of the bottom surface 101 of the housing.

As shown in FIG. 1, the light-emitting assembly includes a lamp panel 500 and a light-pervious cover 600, the light-pervious cover 600 covers the outside of the lamp panel 500, the lamp panel 500 is mounted on the outer surface of the housing 100, the outer surface of the housing 100 is provided with a lamp panel mounting part 103, and the lamp panel 500 is mounted at the lamp panel mounting part 103.

Specifically, the light-pervious cover 600 covers the lamp panel 500 to protect the lamp panel 500 to prevent the lamp panel 500 from being exposed to the outside and being easily damaged by accidental contact, and the light-pervious cover 600 may also achieve a more uniform effect of light to improve an illumination effect.

In some embodiments, the lamp panel mounting part 103 may be arranged on the bottom surface 101 of the housing. At this time, the lamp panel mounting part 103 and the air inlet 1011 cooperate to form a partition setting, as long as the lamp panel mounting part 103 and the air inlet 1011 do not affect each other. When the lamp panel 500 is arranged on the bottom surface 101 of the housing, the light-emitting assembly is arranged in a circumferential direction of the air inlet 1011, that is, the lamp panel mounting part 103 is arranged in the circumferential direction of the air inlet. At this time, the lamp panel 500 is a ring structure, and the lamp panel 500 having the ring structure and the lamp panel mounting part 103 are mounted in cooperation so as to form a light-emitting region and an air inlet region in partition setting on the bottom surface 101 of the housing.

The air inlet 1011 is used for allowing the air to flow into the inside of the housing 100, and the lamp panel mounting part 103 is used for mounting the lamp panel 500. By arranging the lamp panel mounting part 103 around the air inlet 1011, the air inlet 1011 and the lamp panel mounting part 103 form a central air inlet region and an annular mounting region of the lamp panel 500.

It should be noted that the lamp panel mounting part 103 is arranged in a ring structure, but the lamp panel 500 is not necessarily arranged in a ring structure. The lamp panel mounting part 103 is used for mounting the lamp panel 500, that is, the lamp panel 500 may be mounted on the lamp panel mounting part 103, the lamp panel 500 may also be partially mounted on the lamp panel mounting part 103, and the lamp panel 500 may also not be mounted on the lamp panel mounting part 103.

In some embodiments, the lamp panel mounting part 103 is not limited to be mounted on the bottom surface 101 of the housing. The lamp panel mounting part 103 may also be arranged on a side surface 102 of the housing. Of course, the lamp panel mounting part 103 may also be arranged on a top surface. The lamp panel mounting part 103 may be adaptively arranged specifically based on actual scenarios.

It should be noted that the lamp panel mounting part 103 may be simultaneously arranged on the bottom surface 101 of the housing and the side surface 102 of the housing to increase the illumination intensity and the illumination range. The lamp panel mounting part 103 may also be simultaneously arranged on the bottom surface 101 of the housing, the side surface 102 of the housing and the top surface to further increase the illumination intensity and the illumination range.

As shown in FIG. 1 and FIG. 4, the sterilization and disinfection lamp further includes a fan 300, and the fan 300 is used for blowing positive ions and negative ions into the outside environment from the air outlet 1021.

Specifically, the fan 300 is mounted inside the housing 100, the fan 300 is arranged close to the air inlet 1011 relative to an emission head assembly 202 of the plasma generator, the plasma generator discharges the air inside the housing 100 to generate positive ions and negative ions, and the fan 300 blows the positive ions and the negative ions from the air outlet 1021 into the outside environment to sterilize the air of the outside environment.

In some embodiments, the air enters the inside of the housing 100 from the air inlet 1011 on the housing 100 through the fan 300, the air entering the inside of the housing 100 passes through the plasma emitted by the plasma generator, the plasma is provided with positive ions and negative ions, the positive ions and the negative ions sterilize the air inside the housing 100, and under the action of airflow, some positive ions and negative ions diffuse into the air outside the housing 100 through the air outlet 1021 following the air, so as to sterilize the air outside the housing 100. An air outlet surface is arranged along a circumferential direction of an air inlet surface, so that the air from the air inlet 1011 uniformly diffuses to the outside of the housing 100 through the air outlet 1021 under the action of the fan 300, thus increasing the sterilization efficiency.

It should be noted that the plasma will rapidly decay and fail in the temperature environment above 50°, so it is necessary to control the air velocity and air volume of the fan 300 to control the temperature inside the housing 100.

In the present embodiment, the air velocity is 3-5 m/s, and the air volume is greater than 50 cfm.

In the present embodiment, the fan 300 is a backward centrifugal fan which enables the air entering the inside of the housing 100 through the air inlet 1011 of the bottom surface to be discharged out of the housing 100 through the air outlet 1021 on the side surface 102 of the housing.

As shown in FIG. 4, the fan 300 is directly opposite to the air inlet 1011, so that the air from the air inlet 1011 uniformly flows to the surrounding air outlets 1021 through the fan 300.

Specifically, the directly opposite fan 300 and air inlet 1011 may increase the flow efficiency of the air, increase the contact area between the air and the positive ions and negative ions, and increase the rate of the air driving the positive ions and negative ions, thus increasing the sterilization efficiency.

In the present embodiment, the air outlet 1021 is arranged on the side surface 102 of the housing, the side surface 102 of the housing is an air outlet surface so that the side surface 102 of the housing is provided with the air outlet 1021, and one or a plurality of air outlets 1021 may be provided.

In some embodiments, when one air outlet 1021 is provided, the air outlet 1021 is an open structure that extends circumferentially around the side surface 102 of the housing, and the open structure is in the shape of a ring to enable the air entering the inside of the housing 100 to uniformly flow to the outside of the housing 100.

In the present embodiment, when a plurality of air outlets 1021 are provided, two different implementations are provided as follows according to different structure settings of the plurality of air outlets 1021.

In a specific implementation, the air outlets 1021 are arranged at intervals along a circumferential direction of the housing 100, so that the air entering the inside of the housing 100 is blown out through the circumferentially arranged air outlets 1021 located outside the housing 100 to achieve the effect of uniform diffusion to the outside of the housing 100.

In another specific implementation, the air outlets 1021 are arranged at intervals along a height direction of the housing 100, so that the air entering the inside of the housing 100 is blown out through the air outlets 1021 located on the housing 100 to achieve the effect of uniform diffusion to regions of different heights outside the housing 100.

Of course, in a case that there is sufficient area on the side surface 102 of the housing, when a plurality of air outlets 1021 are provided, the air outlets 1021 may be arranged at intervals along the circumferential direction of the housing 100 and also arranged at intervals along the height direction of the housing 100.

It should be noted that when each air outlet 1021 is arranged in a ring structure at this time, the air inside the housing 100 may achieve circumferential uniform diffusion to the outside of the housing 100, and may also achieve uniform diffusion to regions of different heights outside the housing 100.

In the present embodiment, a plurality of air guide ribs are disposed on a surface of the bottom surface 101 of the housing towards the interior of the housing 100, the plurality of air guide ribs are arranged at intervals along the circumferential direction of the air inlet 1011, an air guide duct is formed between two adjacent air guide ribs, the air passing area of the air guide duct gradually increases along the air outlet direction, and the air outlets 1021 of the air guide duct are arranged correspondingly.

Specifically, the air guide ribs are arranged to guide the air entering the inside of the housing 100 through the air inlet 1011, and under the action of the fan 300, the air entering the inside of the housing 100 flows along the surfaces of the air guide ribs and flows to the air outlet 1021.

In some embodiments, after the air entering through the air inlet 1011 enters the inside of the housing 100, the air uniformly flows outward through the air outlet 1021, and the air passing area of the air guide duct gradually increases along the air outlet direction, so that the air entering the inside of the air guide duct uniformly diffuses and then uniformly flows to the outside of the housing 100 through the air outlet 1021.

In some embodiments, the air guide rib is an arc-shaped section and is provided with a first radian, and the first radian is greater than 0° and less than 90°. By setting the air guide rib with a radian, the air inside the housing 100 may carry positive ions and negative ions to be blown out of the housing 100, so as to increase the contact area with the air outside the housing 100. The first radian needs to be less than 90° to prevent the air guide rib from hindering the flow of the air inside the housing 100 out of the housing 100.

In the present embodiment, the plasma generator includes a plasma flyback transformer 201 and an emission head assembly 202, the plasma flyback transformer 201 is mounted on the housing 100, the emission head assembly 202 is arranged on a cavity wall of the sterilization cavity, the plasma flyback transformer 201 is connected to the emission head assembly 202, at least an emission head of the emission head assembly 202 is arranged inside the air guide duct, and at least one emission head assembly 202 is arranged inside at least one air guide duct, wherein one or a plurality of emission head assemblies 202 are provided.

Specifically, when one emission head assembly 202 is provided, the emission head of the emission head assembly 202 is located inside one air guide duct. When a plurality of emission head assemblies 202 are provided, the plurality of emission head assemblies 202 are arranged at intervals along the circumferential direction of the cavity wall of the sterilization cavity, the emission head of each emission head assembly 202 is exposed in the sterilization cavity, and the emission heads of the plurality of emission head assemblies 202 are located inside one air guide duct or respectively located inside different air guide ducts.

In some embodiments, the plasma flyback transformer 201 may be mounted inside the housing 100, and the plasma flyback transformer 201 may also be mounted outside the housing 100, as long as the emission head of the emission head assembly 202 may be located inside the air guide duct.

In some embodiments, the distance between two adjacent emission head assemblies 202 in the plurality of emission head assemblies 202 is the same. This arrangement may avoid interference between two adjacent emission head assemblies 202 to ensure the stable operation of the emission head assemblies 202.

It should be noted that the emission head assembly 202 includes a positive ion emission head and a negative ion emission head, the positive ion emission head is parallel to the negative ion emission head, and an extension direction of the positive ion emission head and the negative ion emission head is perpendicular or horizontal to an air flow direction in the sterilization cavity.

In the present embodiment, the plasma generator includes one of a high-energy plasma generator and a low-energy plasma generator.

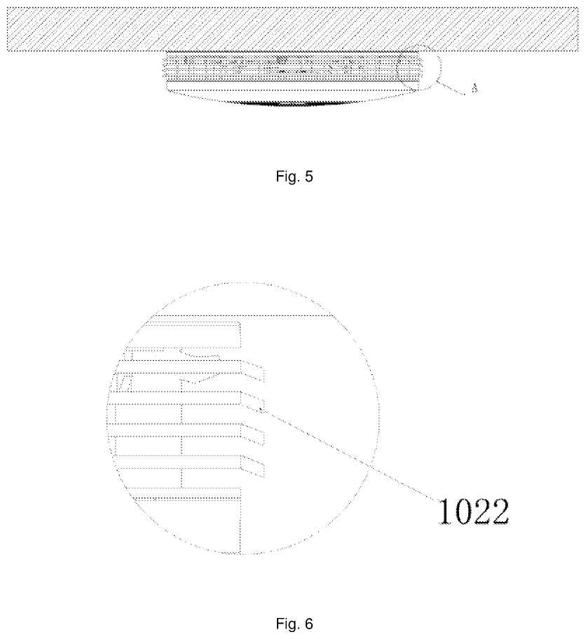

As shown in FIG. 5 and FIG. 6, the air outlet 1021 is provided with an air outlet grid 1022, an air guide surface of the air outlet grid 1022 is obliquely arranged relative to a horizontal plane, an included angle between the air guide surface and the horizontal plane is greater than 0° and less than 90°, and the air guide surface obliquely extends downward along an air outlet direction.

Specifically, by setting the air outlet grid 1022 with an angle, the air flowing out from the air outlet 1021 diffuses into the air in a certain radian under the guidance of the air outlet grid 1022, thus increasing the contact area between the positive ions and negative ions flowing out from the air outlet 1021 and the air outside the housing 100 to enhance the sterilization efficiency.

In some embodiments, by setting the air outlet grid 1022 with different angles, the angle of the air flowing out from the air outlet 1021 may be adjusted to adapt to different application scenarios, thus increasing the practicality and enhancing the user experience.

In some embodiments, the angle of the air outlet grid 1022 may be adjusted to adjust the angle of the air flowing out from the air outlet 1021. During adjusting, the angle of the air outlet grid 1022 may be adjusted according to the number of people and the positions of people. The air direction and air velocity of the air flowing out from the air outlet 1021 may be adjusted by adjusting the angle of the air outlet grid 1022. When the air direction is changed by adjusting the angle of the air outlet grid 1022, the air flowing out from the air outlet 1021 flows to regions of people to sterilize and disinfect the air of the regions of people, thus ensuring that the air that people breathe is sterilized and disinfected to achieve healthy breathing of people. When the air velocity is changed by adjusting the angle of the air outlet grid 1022, the air outlet grid 1022 may be adaptively adjusted according to human perception, thus enhancing the user experience. Furthermore, the angle of the air outlet grid 1022 may also be adjusted according to the size of the space where the sterilization and disinfection lamp is located, so that the sterilization and disinfection lamp may sterilize and disinfect the air inside the space to the maximum extent to improve the sterilization effect.

In some embodiments, the air guide surface obliquely extends downward along the air outlet direction, so that the positive ions and negative ions flowing out from the air outlet 1021 flow downward in multiple parabolas.

As shown in FIG. 1, the sterilization and disinfection lamp further includes a filter sieve 700, and the filter sieve 700 is detachably arranged. The filter sieve 700 is arranged at the air inlet 1011, or the filter sieve 700 and the air inlet 1011 may be arranged at an interval to filter the air entering the inside of the housing 100.

Specifically, when the filter sieve 700 is arranged at the air inlet 1011, the air entering the inside of the housing 100 is filtered for the first time by arranging the detachable filter sieve to avoid affecting the cleanliness of the inside of the housing 100. When the filter sieve needs to be replaced, the filter sieve may be disassembled and mounted according to needs at any time, and the operation is convenient.

In some embodiments, when the filter sieve 700 and the air inlet 1011 are arranged at an interval, the air inlet surface is provided with a convex rib 104 extending towards the air inlet direction, the convex rib 104 encloses an air inlet channel, an inlet of the air inlet channel is the air inlet 1011, the filter sieve 700 is arranged in the air inlet channel, and the filter sieve arranged at an interval may avoid affecting the flow rate of the air from the air inlet 1011.

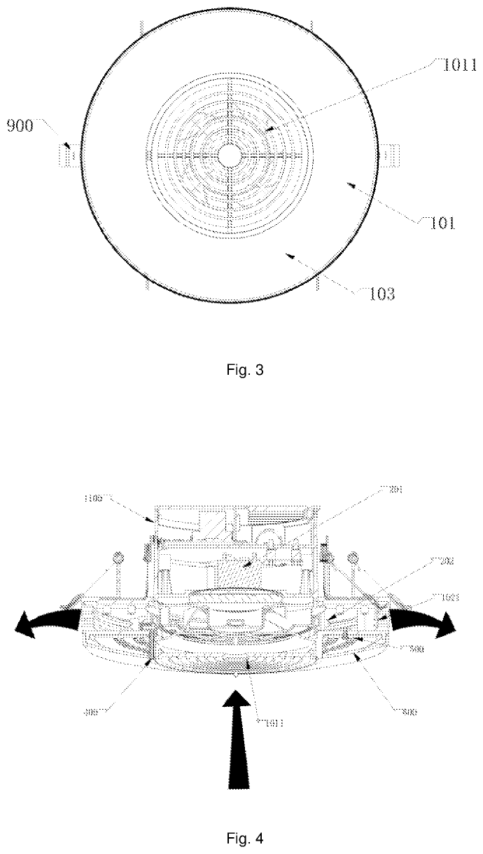

As shown in FIG. 1 and FIG. 3, the sterilization and disinfection lamp further includes a junction box 1100 arranged on an outer side of the housing 100, the junction box 1100 is arranged on a surface of the housing 100 away from the air inlet 1011, a power supply 1200, a circuit module and a switch 1300 are arranged inside the junction box 1100, the power supply 1200 is electrically connected to the circuit module through the switch 1300, and the circuit module is electrically connected to the fan 300 and/or the plasma generator and/or the light-emitting assembly.

Specifically, the junction box 1100 is arranged on the surface of the housing 100 away from the air inlet 1011 to prevent the air inside the housing 100 from entering the inside of the junction box 1100 to increase the flow rate of the air, thus increasing the sterilization efficiency.

It should be noted that circuits between the plasma generator and the light-emitting assembly are independent, the light-emitting assembly may be independently controlled to work, and the plasma generator may also be independently controlled to work.

As shown in FIG. 1, the sterilization and disinfection lamp further includes an indication lamp 400, the indication lamp 400 is electrically connected to the circuit module, and at least part of the indication lamp 400 is located on the air inlet surface.

Specifically, by arranging the indication lamp 400, it is convenient to observe the working state, and the working state of the plasma generator may be distinguished by the indication lamps 400 of different colors.

In some embodiments, the indication lamp 400 may be attached to the air inlet surface or may be attached to the outer circumferential surface of the convex rib 104 for facilitating observation.

In some embodiments, the indication lamp 400 is not limited to be mounted in a manner of attaching and may also be mounted in a manner of a mounting post 401, and the structure of the mounting post 401 may be adaptively adjusted according to mounting requirements of the indication lamp 400 to ensure stable mounting of the indication lamp 400.

It should be noted that the indication lamp 400 is not limited to be mounted on the air inlet surface and may also be mounted on the air outlet surface. When the indication lamp 400 is mounted on the air outlet surface, the indication lamp 400 may be arranged along the circumferential direction of the air outlet surface and may also be arranged along the height direction of the air outlet surface for facilitating observation.

As shown in FIG. 1 and FIG. 3, the combined lamp further includes a connecting structure, the connecting structure includes a plurality of connecting pieces arranged at intervals along the circumferential direction of the housing 100 of the sterilization and disinfection lamp, the connecting piece is detachably mounted on the surface of the housing 100 away from the air inlet 1011 of the sterilization and disinfection lamp, and the connecting piece includes a spring assembly 900 and a torsion spring assembly 800 which are used in cooperation.

Specifically, the sterilization and disinfection lamp is mounted in a manner of hanging through the spring assembly 900 and the torsion spring assembly 800 which are used in cooperation.

In some embodiments, the spring assembly 900 includes a spring support and a spring which are connected, the torsion spring assembly 800 includes a torsion spring support and a torsion spring which are connected, the spring support and the torsion spring support are oppositely arranged at an interval and are mounted and fixed by the spring and the torsion spring, and the spring and the torsion spring are used in cooperation to increase the mounting stability.

The sterilization and disinfection lamp includes at least one of a table lamp, a hanging lamp, a floor lamp, a ceiling lamp, a semi-ceiling lamp, a semi-hanging lamp, a wall lamp and a mirror lamp.

### Embodiment 2

Different from Embodiment 1, in the present embodiment, the air outlet 1021 is located on the bottom surface 101 of the housing, and the air inlet 1011 is arranged on the side surface 102 of the housing.

Specifically, the air inlet 1011 is arranged in the circumferential direction of the side surface of the housing, the air outlet 1021 is located in a center position of the bottom surface 101 of the housing, the fan 300 is a reverse centrifugal fan 300, and the reverse centrifugal fan 300 ensures that the air enters the inside of the housing 100 through the air inlet 1011 arranged on the side surface 102 of the housing, passes through the emission head assembly of the plasma generator, and then is blown to the outside environment through the air outlet 1021 on the bottom surface 101 of the housing.

In the present embodiment, when a plurality of air inlets 1011 are provided, two different implementations are provided as follows according to different structure settings of the plurality of air inlets 1011.

In a specific implementation, the air inlets 1011 are arranged at intervals along the circumferential direction of the housing 100, so that the air enters the inside of the housing 100 through the circumferentially arranged air inlets 1011 located outside the housing 100 to achieve the effect of uniform intake to the inside of the housing 100.

In another specific implementation, the air inlets 1011 are arranged at intervals along the height direction of the housing 100.

Of course, in a case that there is sufficient area on the side surface 102 of the housing, when a plurality of air inlets 1011 are provided, the air inlets 1011 may be arranged at intervals along the circumferential direction of the housing 100 and also arranged at intervals along the height direction of the housing 100.

### Embodiment 3

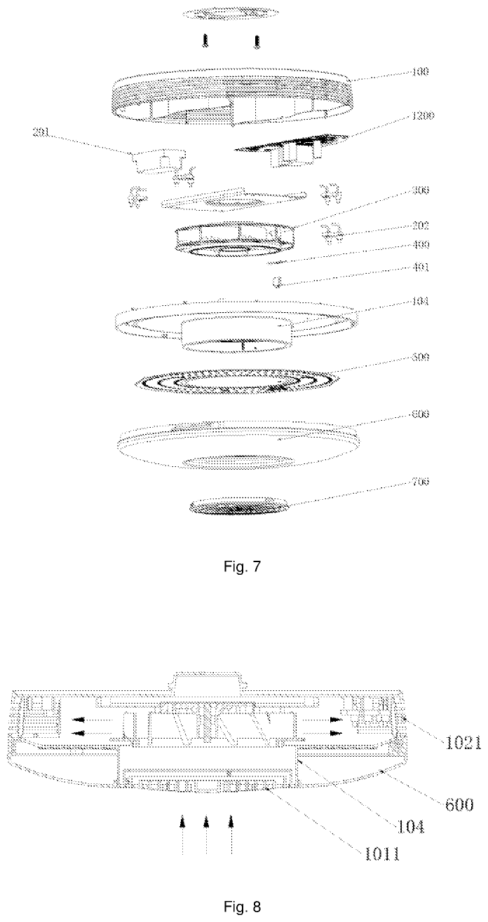

Different from Embodiment 1, as shown in FIG. 7 to FIG. 8, the sterilization and disinfection lamp in the present embodiment is not provided with the junction box 1100, a fixed plate is arranged inside the sterilization and disinfection lamp, and the fan 300, the power supply 1200, the circuit module, the switch 1300 and the plasma generator are all mounted on the fixed plate.

### Embodiment 4

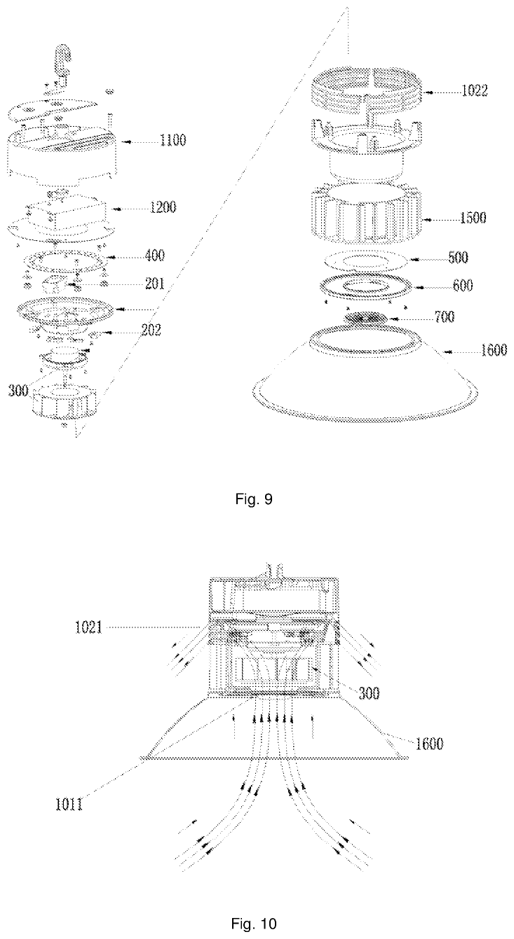

Different from Embodiment 1, as shown in FIG. 9 to FIG. 10, the combined lamp in the present embodiment further includes a radiator 1500 and a lampshade 1600.

Specifically, the gas temperature inside the housing 100 may be less than or equal to 50° by arranging the radiator 1500 to avoid affecting the plasma sterilization effect due to excessive temperature, wherein the area of the radiator 1500 may be adaptively adjusted according to actual situations so that the gas temperature inside the housing 100 may be less than or equal to 50° by the radiator 1500.

In the present embodiment, two different implementations are provided as follows according to different structures of the radiator 1500.

In a specific implementation not illustrated, the radiator 1500 is arranged in a ring structure, and the radiator 1500 is padded between the lamp panel 500 and the air inlet surface to perform heat conduction treatment on the heat emitted by the lamp panel 500, thus accelerating heat dissipation, and prolonging the service life of the lamp panel 500.

In a specific implementation shown in the figures, the radiator 1500 is arranged in a cylinder structure, and the air inlet channel of the sterilization and disinfection lamp passes through the radiator 1500 to sleeve the radiator 1500 on the air inlet channel, so as to perform heat conduction treatment on the heat emitted by the lamp panel 500 and the indication lamp 400, thus accelerating heat dissipation, and prolonging the service life of the lamp panel 500 and the indication lamp 400.

As shown in FIG. 9 and FIG. 10, the lampshade 1600 is a horn structure, the lampshade 1600 is provided with a first opening and a second opening, the diameter of the second opening is greater than the diameter of the first opening, the second opening is located below the first opening, the sterilization and disinfection lamp is located in the lampshade 1600, and the air inlet 1011 of the sterilization and disinfection lamp faces the second opening.

Specifically, the lampshade 1600 having the horn structure is provided with the function of converging and guiding light to avoid the problem of poor illumination effect caused by excessive dispersion of light.

As shown in FIG. 9, in the present implementation, a mounting plate is arranged on a top surface of the junction box 1100, and a connecting structure is arranged on the mounting plate, so that the sterilization and disinfection lamp may be mounted in a manner of hanging through the connecting structure.

Specifically, the connecting structure includes at least one of a hanging hook, a suspension chain and a clamp hook, and the connecting structure is arranged on the surface of the sterilization and disinfection lamp away from the air inlet surface.

It should be noted that the connecting structure needs to ensure stable mounting of the sterilization and disinfection lamp.

### Embodiment 5

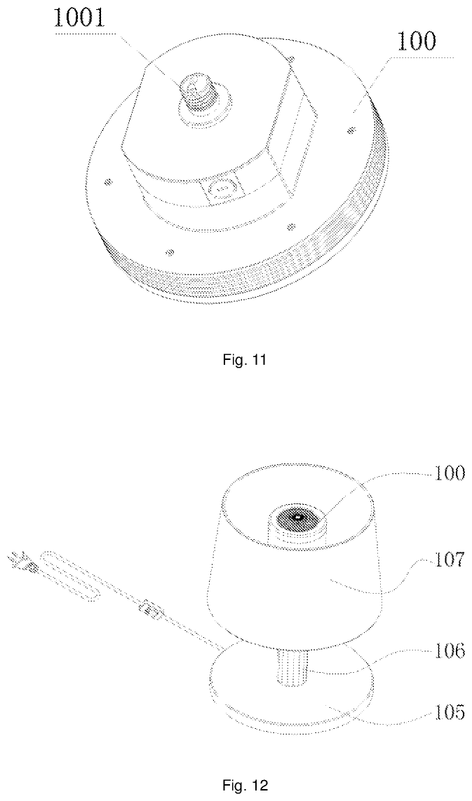

Different from Embodiment 1, in the present embodiment, as shown in FIG. 11, a mounting head 1001 of the housing 100 is provided with a first connecting structure, the sterilization and disinfection lamp further includes a mounting base, the mounting base is provided with a second connecting structure in cooperation with the first connecting structure, and the first connecting structure and the second connecting structure may be connected in place to supply power to the sterilization and disinfection lamp.

Specifically, an outer surface of the mounting head 1001 is provided with a first mounting thread, the mounting base is provided with a mounting groove, the mounting groove is provided with a second mounting thread in cooperation with the first mounting thread, the mounting base supplies power to the sterilization and disinfection lamp after the first mounting thread and the second mounting thread are connected in place, the first mounting thread serves as the first connecting structure, and the second mounting thread serves as the second connecting structure. Rotational connection is performed in a manner similar to bulb mounting, and electrical connection is implemented.

Of course, the mounting head 1001 may also be connected to the mounting base in a form of clamping. An outer surface of the mounting head 1001 is provided with a first clamping structure, the mounting base is provided with a second clamping structure in cooperation with the first clamping structure, the mounting base supplies power to the sterilization and disinfection lamp after the first clamping structure and the second clamping structure are clamped in place, the first clamping structure serves as the first connecting structure, and the second clamping structure serves as the second connecting structure.

Of course, the mounting head 1001 may also be fixedly connected to the mounting base to ensure the tightness of connection between the mounting head 1001 and the mounting base, wherein the first connecting structure is fixedly connected to the second connecting structure.

Of course, a wire connection may also be adopted between the mounting head 1001 and the mounting base, and the first connection structure is connected to the second connection structure in a wire manner, so as to ensure stability of connection and power supply.

### Embodiment 6

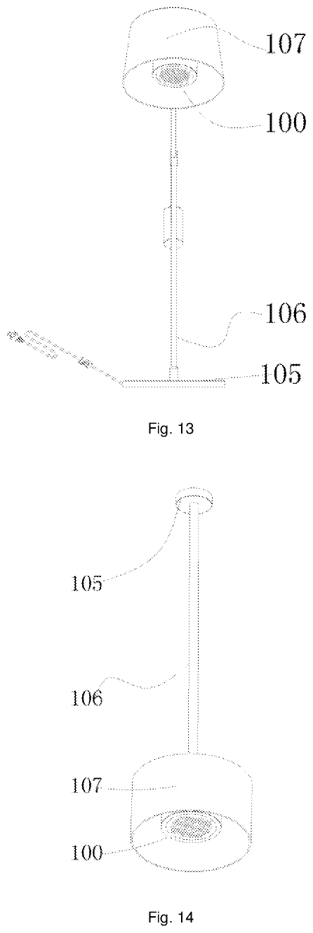

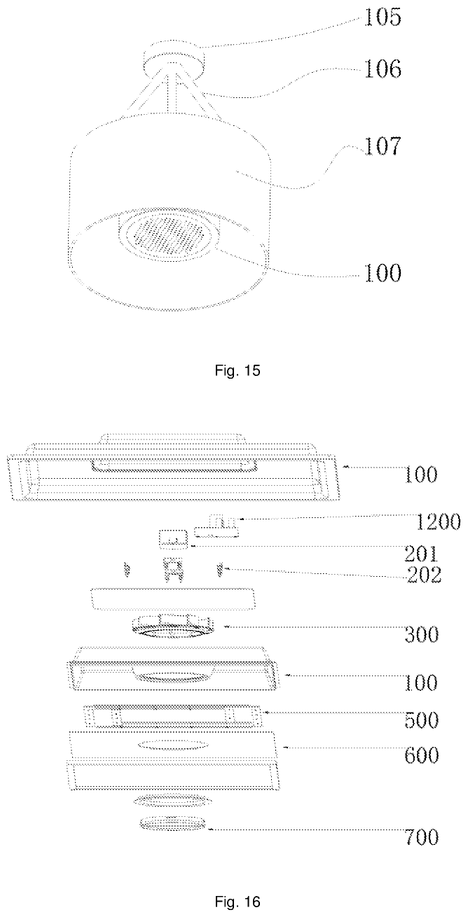

Different from Embodiment 1, in the present embodiment, as shown in FIG. 12 to FIG. 15, the sterilization and disinfection lamp further includes a support post 106 and a chassis 105, one end of the support post 106 is connected to the mounting base, the support post 106 is provided with a hollow structure, at least part of wires are accommodated in the support post 106, and the other end of the support post 106 is connected to the chassis 105.

In the present embodiment, the chassis 105 may be mounted on a wall to enable the sterilization and disinfection lamp hang on the wall, or the chassis 105 may be placed on a table top to enable the sterilization and disinfection lamp to stand upright.

Specifically, when the chassis 105 is mounted on a wall, the sterilization and disinfection lamp is a ceiling lamp or a hanging lamp. When the chassis 105 is placed on a table top, the sterilization and disinfection lamp is a table lamp or a floor lamp.

In some embodiments, the sterilization and disinfection lamp may be in a form of air intake on the bottom surface and air discharge on the side surface, or may be in a form of air intake on the side surface and air discharge on the bottom surface.

In the present embodiment, the sterilization and disinfection lamp further includes a cover body 107, the cover body 107 is connected to the mounting base or the support post 106, and the light-emitting assembly is located in the cover body 107 to realize protection of the sterilization and disinfection lamp and ensure uniform diffusion of light.

In some embodiments, the sterilization and disinfection lamp further includes a support rod 108, and the support rod 108 is connected to the chassis 105 and the cover body 107 to enhance the stability of connection.

### Embodiment 7

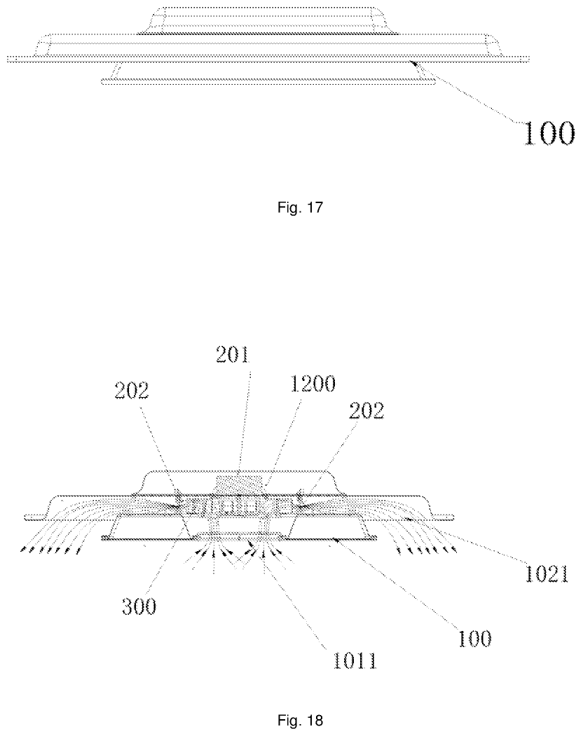

Different from Embodiment 1, as shown in FIG. 16 to FIG. 18, in the present embodiment, the air outlet 1021 is located on the bottom surface 101 of the housing, and at this time, the air inlet 1011 and the air outlet 1021 are located on the same plane.

Specifically, the bottom surface 101 of the housing is provided with the air outlet 1021, and the air outlet 1021 is arranged around the circumferential direction of the air inlet 1011, so that the air discharged from the air outlet 1021 is discharged to the outside region of the housing 100 along the circumferential direction of the air inlet 1011.

In some embodiments, the housing 100 is further provided with an air guide channel, and the air entering the inside of the housing 100 flows to the bottom surface 101 of the housing under the action of the air guide channel and is discharged out of the housing 100 through the air outlet 1021 on the bottom surface.

In the present embodiment, the light-emitting assembly is mounted on the bottom surface 101 of the housing, and the lamp panel 500 of the light-emitting assembly is arranged along the circumferential direction of the air inlet 1011. At this time, the air inlet 1011 and the air outlet 1021 are arranged at an interval to form a mounting region on the bottom surface 101 of the housing, and the light-emitting assembly is mounted in the mounting region.

In the present embodiment, the sterilization and disinfection lamp is a square lamp or a circular lamp.

### Embodiment 8

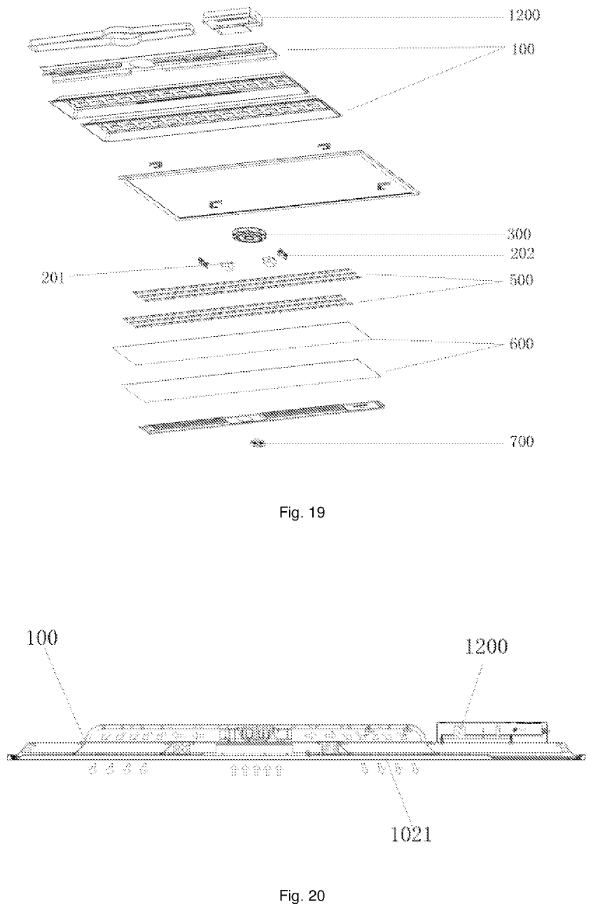

Different from Embodiment 7, as shown in FIG. 19 to FIG. 20, in the present embodiment, the lamp panel 500 is provided with a strip structure, and the power supply 1200 is located on the outer surface of the housing 100.

### Embodiment 9

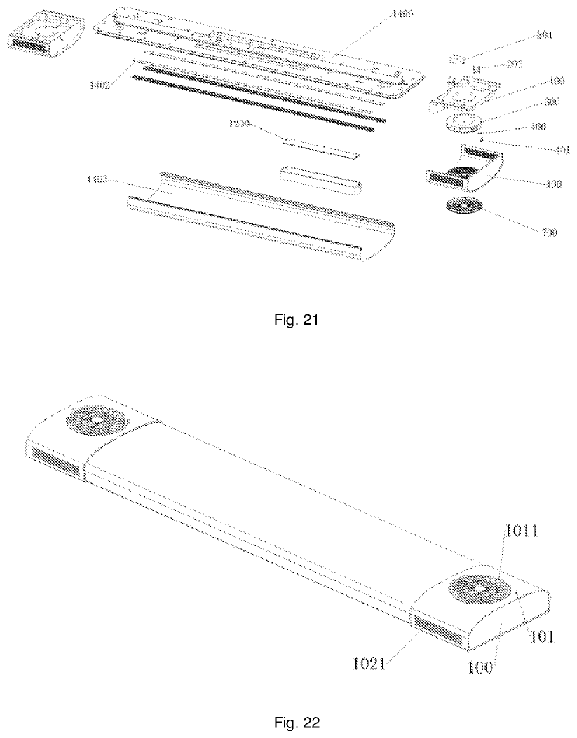

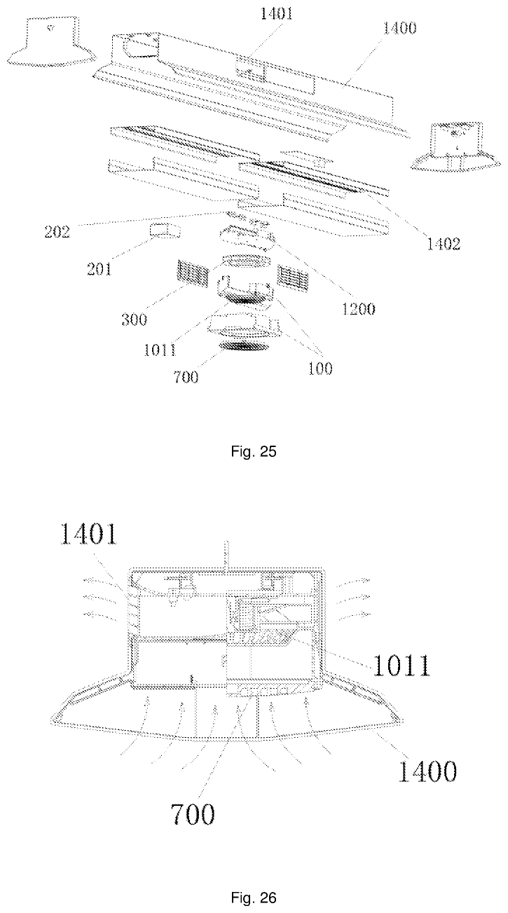

The disclosure further provides a combined lamp. As shown in FIG. 21 to FIG. 26, the combined lamp includes the above sterilization and disinfection lamp and an illuminating lamp 1400, and the illuminating lamp 1400 is connected to the sterilization and disinfection lamp. At this time, the sterilization and disinfection lamp and the illuminating lamp 1400 may emit light simultaneously, so that the light emitting efficiency is increased, and sterilization and disinfection may also be implemented at the same time.

Specifically, the illuminating lamp 1400 includes a lamp housing 1403 and a light source 1402 arranged inside the lamp housing 1403, and the lamp housing 1403 is detachably connected to the housing 100 of the sterilization and disinfection lamp.

In some embodiments, the lamp housing 1403 is provided with an air vent 1401, the air flowing out from the air outlet 1021 on the housing 100 of the sterilization and disinfection lamp may directly communicate with the outside environment, and the air flowing out from the air outlet 1021 on the housing 100 of the sterilization and disinfection lamp may also be discharged to the outside environment through the air vent 1401 on the lamp housing 1403.

In some embodiments, the combined lamp includes at least one light-emitting group, that is, one light-emitting group may be provided, or a plurality of light-emitting groups may cooperate to form the combined lamp, and the plurality of light-emitting groups may be connected by splicing. The light-emitting group is composed of a sterilization and disinfection lamp unit and an illuminating lamp unit, wherein each sterilization and disinfection lamp unit includes one or a plurality of sterilization and disinfection lamps, and each illuminating lamp unit includes one or a plurality of illuminating lamps 1400.

In some embodiments, the splicing manner may be one of insertion, bonding, screw connection, and the like.

In the present embodiment, three different implementations are provided as follows according to different mounting forms of the sterilization and disinfection lamp unit and the illuminating lamp unit in the light-emitting group.

In a specific implementation not illustrated, the light-emitting group includes one sterilization and disinfection lamp unit and one illuminating lamp unit. When a plurality of light-emitting groups are provided, the plurality of light-emitting groups are spliced sequentially.

Specifically, the light-emitting group may include one sterilization and disinfection lamp connected to a plurality of illuminating lamps 1400, or may include one illuminating lamp 1400 connected to a plurality of sterilization and disinfection lamps, and the number of the sterilization and disinfection lamp may be the same or different from the number of the illuminating lamp 1400.

In a specific implementation shown in FIG. 21 to FIG. 22, the light-emitting group includes two sterilization and disinfection lamp units and one illuminating lamp unit, and the two sterilization and disinfection lamp units are respectively arranged at both ends of the illuminating lamp unit. When a plurality of light-emitting groups are provided, the plurality of light-emitting groups are spliced sequentially.

Specifically, one or a plurality of sterilization and disinfection lamps may be provided in the two sterilization and disinfection lamp units at both ends of the illuminating lamp unit. When a plurality of sterilization and disinfection lamps are provided in both sterilization and disinfection lamp units at both ends of the illuminating lamp unit, the number of sterilization and disinfection lamps at both ends may be the same or different, and the illuminating lamp unit may include one illuminating lamp 1400 or a plurality of illuminating lamps 1400.

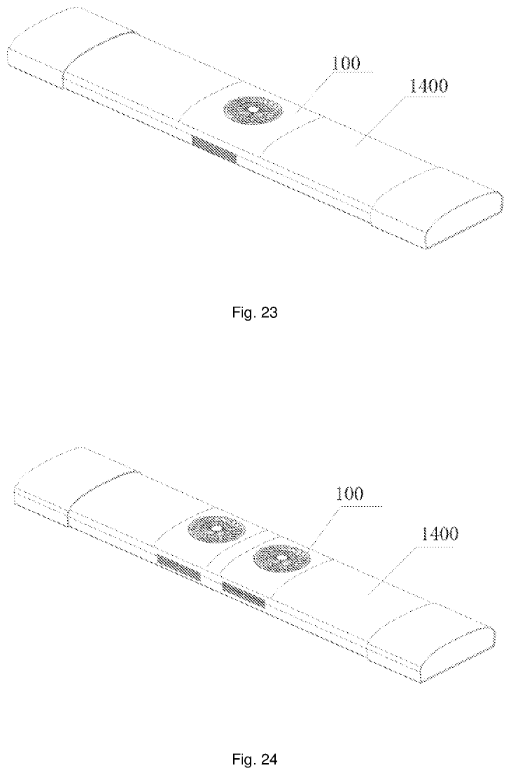

In a specific implementation shown in FIG. 23 to FIG. 26, the light-emitting group includes one sterilization and disinfection lamp unit and two illuminating lamp units, and the two illuminating lamp units are respectively arranged at both ends of the sterilization and disinfection lamp unit. When a plurality of light-emitting groups are provided, the plurality of light-emitting groups are spliced sequentially.

Specifically, the sterilization and disinfection lamp unit between two illuminating lamp units may include one sterilization and disinfection lamp or a plurality of sterilization and disinfection lamps. Each of the two illuminating lamp units at both ends may include one or a plurality of illuminating lamps 1400, and the number of the illuminating lamps 1400 in the two illuminating lamp units may be the same or different.

### Embodiment 10

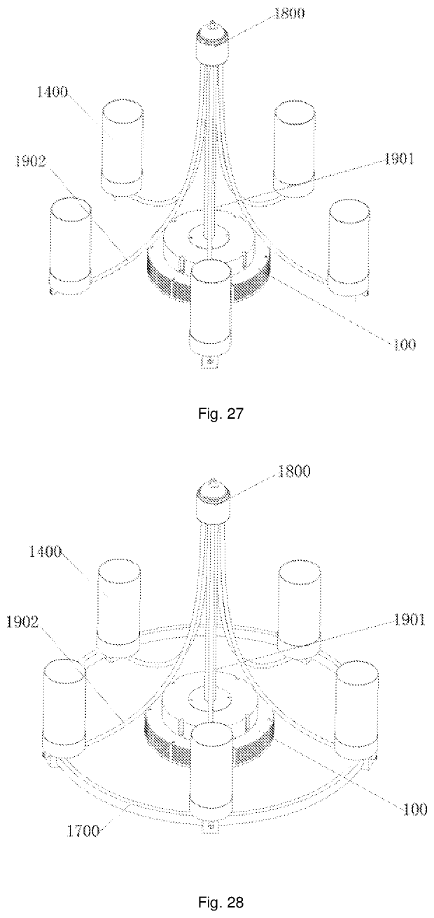

Different from Embodiment 9, as shown in FIG. 27 and FIG. 28, the combined lamp further includes a connector 1800 and a connecting post 1901, one end of the connecting post 1901 is connected to the top surface of the housing 100 of the sterilization and disinfection lamp, the other end of the connecting post 1901 is connected to the connector 1800, the illuminating lamp 1400 is connected to the sterilization and disinfection lamp or connected to the connecting post 1901 or connected to the connector 1800, the connector 1800 may be mounted on a wall to enable the sterilization and disinfection lamp hang on the wall, or the connector 1800 may be placed on a table top to enable the sterilization and disinfection lamp to stand upright.

Specifically, when the connector 1800 is mounted on a wall, the combined lamp is a hanging lamp or a suspended lamp. When the connector 1800 is mounted on a table top or ground, the combined lamp is a table lamp or a floor lamp.

In the present embodiment, the combined lamp includes one sterilization and disinfection lamp and a plurality of illuminating lamps 1400, and the plurality of illuminating lamps 1400 are arranged at intervals along the circumferential direction of the sterilization and disinfection lamp.

In the present embodiment, two different implementations are provided as follows according to different connecting structures of the illuminating lamp 1400.

In a specific implementation shown in the figures, the combined lamp further includes a plurality of connecting rods 1902, a connector 1800 and a connecting post 1901, one end of the connecting post 1901 is connected to the top surface of the housing 100 of the sterilization and disinfection lamp, the other end of the connecting post 1901 is connected to the connector 1800, one end of the connecting rod 1902 is connected to the illuminating lamp 1400, and the other end of the connecting rod 1902 is connected to the connector 1800.

In a specific implementation shown in the figures, the combined lamp further includes a connector 1800, a lamp ring 1700, a connecting post 1901 and a plurality of connecting rods 1902, wherein a plurality of illuminating lamps 1400 are connected through the lamp ring 1700, the connecting rods 1902 and the illuminating lamps 1400 are arranged correspondingly, one end of the connecting rod 1902 is connected to the illuminating lamp 1400, and the other end of the connecting rod 1902 is connected to the connector 1800; and one end of the connecting post 1901 is connected to the top surface of the housing 100 of the sterilization and disinfection lamp, and the other end of the connecting post 1901 is connected to the connector 1800, thus enhancing the aesthetics of the lamp and also increasing the illumination range of the lamp.

### Embodiment 11

The disclosure further provides a lamp. The difference between the present embodiment and Embodiment 9 is that in the present embodiment, the sterilization and disinfection lamp is replaced with a sterilization module to form a new lamp. In the present embodiment, the sterilization module is not provided with a light-emitting assembly compared to the sterilization and disinfection lamp.

Specifically, the lamp includes the above housing 100, the fan of the above sterilization and disinfection lamp and the above plasma generator, and the housing 100, the fan and the plasma generator cooperate to form a sterilization module. The lamp further includes illuminating lamps 1400 connected to the sterilization module, the sterilization module is provided with a sterilization function, the illuminating lamp 1400 is provided with an illumination function, and the illuminating lamp 1400 and the sterilization module cooperate to achieve illumination and sterilization functions.

In some embodiments, the illuminating lamp 1400 includes a lamp housing 1403 and a light source 1402 arranged inside the lamp housing 1403, and the lamp housing 1403 is detachably connected to the housing 100.

In some embodiments, the lamp includes at least one light-emitting group, that is, one light-emitting group may be provided, or a plurality of light-emitting groups may cooperate to form the lamp, and the plurality of light-emitting groups may be connected by splicing. The light-emitting group is composed of a sterilization module unit and an illuminating lamp unit, wherein each sterilization module unit includes one or a plurality of sterilization modules, and each illuminating lamp unit includes one or a plurality of illuminating lamps 1400.

In some embodiments, the splicing manner may be one of insertion, bonding, screw connection, and the like.

In the present embodiment, three different implementations are provided as follows according to different mounting forms of the sterilization module unit and the illuminating lamp unit in the light-emitting group.

In a specific implementation not illustrated, the light-emitting group includes one sterilization module unit and one illuminating lamp unit. When a plurality of light-emitting groups are provided, the plurality of light-emitting groups are spliced sequentially.

Specifically, the light-emitting group may include one sterilization module connected to a plurality of illuminating lamps 1400, or may include one illuminating lamp 1400 connected to a plurality of sterilization modules.

In a specific implementation not illustrated, the light-emitting group includes two sterilization module units and one illuminating lamp unit, and the two sterilization module units are respectively arranged at both ends of the illuminating lamp unit. When a plurality of light-emitting groups are provided, the plurality of light-emitting groups are spliced sequentially.

Specifically, one or a plurality of sterilization modules may be provided in the two sterilization module units at both ends of the illuminating lamp unit. When a plurality of sterilization modules are provided in both sterilization module units at both ends of the illuminating lamp unit, the number of sterilization modules at both ends may be the same or different, and the illuminating lamp unit may include one illuminating lamp 1400 or a plurality of illuminating lamps 1400.

In a specific implementation not illustrated, the light-emitting group includes one sterilization module unit and two illuminating lamp units, and the two illuminating lamp units are respectively arranged at both ends of the sterilization module unit. When a plurality of light-emitting groups are provided, the plurality of light-emitting groups are spliced sequentially.

Specifically, the sterilization module unit between two illuminating lamp units may include one sterilization module or a plurality of sterilization modules. Each of the two illuminating lamp units at both ends may include one or a plurality of illuminating lamps 1400, and the number of the illuminating lamps 1400 in the two illuminating lamp units may be the same or different.

### Embodiment 12

Different from Embodiment 11, in the present embodiment, the lamp further includes a connector 1800 and a connecting post 1901, one end of the connecting post 1901 is connected to the top surface of the housing 100 of the sterilization module, the other end of the connecting post 1901 is connected to the connector 1800, the illuminating lamp 1400 is connected to the sterilization module or connected to the connecting post 1901 or connected to the connector 1800, the connector 1800 may be mounted on a wall to enable the sterilization module hang on the wall, or the connector 1800 may be placed on a table top to enable the sterilization module to stand upright.

Specifically, when the connector 1800 is mounted on a wall, the lamp is a hanging lamp or a suspended lamp. When the connector 1800 is mounted on a table top or ground, the lamp is a table lamp or a floor lamp.

In the present embodiment, the lamp includes one sterilization module and a plurality of illuminating lamps 1400, and the plurality of illuminating lamps 1400 are arranged at intervals along the circumferential direction of the sterilization module.

In the present embodiment, two different implementations are provided as follows according to different connecting structures of the illuminating lamp 1400.

In a specific implementation not illustrated, the lamp further includes a plurality of connecting rods 1902, a connector 1800 and a connecting post 1901, one end of the connecting post 1901 is connected to the top surface of the housing 100 of the sterilization module, the other end of the connecting post 1901 is connected to the connector 1800, one end of the connecting rod 1902 is connected to the illuminating lamp 1400, and the other end of the connecting rod 1902 is connected to the connector 1800.

In a specific implementation not illustrated, the lamp further includes a connector 1800, a lamp ring 1700, a connecting post 1901 and a plurality of connecting rods 1902, wherein a plurality of illuminating lamps 1400 are connected through the lamp ring 1700, the connecting rods 1902 and the illuminating lamps 1400 are arranged correspondingly, one end of the connecting rod 1902 is connected to the illuminating lamp 1400, and the other end of the connecting rod 1902 is connected to the connector 1800; and one end of the connecting post 1901 is connected to the top surface of the housing 100 of the sterilization module, and the other end of the connecting post 1901 is connected to the connector 1800, thus enhancing the aesthetics of the lamp and also increasing the illumination range of the lamp.

In the present embodiment, the types of illuminating lamps 1400 may be diverse. For embodiment, the illuminating lamp includes one of a ceiling lamp, a hanging lamp, a floor lamp, a table lamp, a wall lamp, a recessed lamp, a projection lamp and a track lamp. The illuminating lamp 1400 and the sterilization module are mounted in cooperation to achieve illumination and sterilization functions. The illuminating lamp 1400 is provided with a first connecting part, the sterilization module is provided with a second connecting part, the first connecting part and the second connecting part may be independently mounted on a wall or a table top, the first connecting part may also be connected to the second connecting part, and at least one of the first connecting part and the second connecting part is mounted on a wall or a table top.

It should be noted that lamps are not limited to the above types. The illuminating lamp 1400 may also be a lamp having another structure for achieving an illumination function.

From the above description, it may be seen that the above embodiments of the disclosure achieve the following technical effects:

1. The use of the plasma sterilization technology may effectively achieve man-machine symbiosis without affecting the human body.

2. The plasma generator generates positive ions and negative ions to sterilize the air inside the housing 100, and the positive ions and negative ions diffuse with the air to the outside region of the housing 100 to achieve sterilization, thus increasing the sterilization efficiency.

3. The sterilization and disinfection lamp is suitable for mounting requirements in different scenarios, thus extending the application range.

4. By applying various ventilation methods such as air intake on the bottom surface and air discharge on the side surface as well as air intake on the bottom surface and air discharge on the bottom surface, the contact area between the positive ions and negative ions and the air is increased.

5. The housing 100 is provided with the lamp panel mounting part 103, the lamp panel 500 is mounted on the housing 100 to form a sterilization and disinfection lamp, and the sterilization and disinfection lamp is provided with an illumination function and a sterilization function.

It is apparent that the embodiments described above are only a part of the embodiments of the disclosure, but are not all of the embodiments. All other embodiments obtained by those of ordinary skill in the art based on the embodiments in the disclosure without creative efforts shall fall within the scope of protection of the disclosure.

It should be noted that the terms used here are only used for describing specific implementations, but are not intended to limit the exemplary implementations according to the disclosure. As used here, unless explicitly stated in the context, the singular form is also intended to include the plural form. In addition, it should also be understood that when the terms “include” and/or “include” are used in the specification, they indicate the presence of features, steps, operations, devices, assemblies, and/or combinations thereof.

It should be noted that the terms such as “first” and “second” in the specification, claims and above accompanying drawings of the disclosure are used for distinguishing similar objects instead of describing a particular sequence or a precedence order. It should be understood that the data used in this way may be interchanged in appropriate circumstances, so that the implementations of the disclosure described here may be implemented in a sequence other than those illustrated or described here.

The above descriptions are merely preferred embodiments of the disclosure, but are not intended to limit the disclosure. For those skilled in the art, the disclosure may have various modifications and variations. Any modifications, equivalent replacements, improvements, and the like made within the spirit and principle of the disclosure shall be included within the scope of protection of the disclosure.

1. A sterilization and disinfection lamp, comprising:
a housing, wherein the housing is provided with a sterilization cavity, and an air inlet and an air outlet, which communicate with the sterilization cavity;
a light-emitting assembly, which is mounted on an outer surface of the housing; and
a plasma generator, at least part of which is accommodated in the sterilization cavity and generates positive ions and negative ions, wherein at least some positive ions and negative ions are combined in the sterilization cavity to sterilize and disinfect air flowing into the sterilization cavity through the air inlet, and at least some other positive ions and negative ions flow into the outside environment through the air outlet and are combined, so as to sterilize and disinfect the air of the outside environment.

2. The sterilization and disinfection lamp according to claim 1, wherein the air inlet or the air outlet is located at a center of a bottom surface of the housing.

3. The sterilization and disinfection lamp according to claim 1, wherein
the air inlet is arranged on the bottom surface of the housing, one or a plurality of air inlets are provided, the plurality of air inlets are arranged in a ring structure in cooperation, and adjacent air inlets are arranged at an interval.

4. The sterilization and disinfection lamp according to claim 1, wherein the air inlet is arranged on a side surface of the housing, one or a plurality of air inlets are provided, and when a plurality of air inlets are provided,
the air inlets are arranged at intervals along a circumferential direction of the housing; and/or
the air inlets are arranged at intervals along a height direction of the housing.

5. The sterilization and disinfection lamp according to claim 1, wherein the air outlet is arranged on the bottom surface of the housing, one or a plurality of air outlets are provided, and when a plurality of air outlets are provided, the plurality of air outlets are arranged in a ring structure in cooperation, and adjacent air outlets are arranged at an interval.

6. The sterilization and disinfection lamp according to claim 1, wherein the air outlet is arranged on the side surface of the housing, one or a plurality of air outlets are provided, and when a plurality of air outlets are provided,
the air outlets are arranged at intervals along the circumferential direction of the housing; and/or
the air outlets are arranged at intervals along the height direction of the housing.

7. The sterilization and disinfection lamp according to claim 1, wherein
the light-emitting assembly is arranged on the bottom surface of the housing; and/or
the light-emitting assembly is arranged in a circumferential direction of the air inlet and/or the air outlet on the bottom surface of the housing.

8. The sterilization and disinfection lamp according to claim 1, wherein
the sterilization and disinfection lamp further comprises a fan, the fan is a centrifugal fan, the air inlet is arranged on the bottom surface of the housing, the air outlet is arranged on the side surface of the housing and/or the air outlet is arranged on the bottom surface of the housing, the fan is used for blowing the positive ions and the negative ions into the outside environment from the air outlet, the fan is directly opposite to the air inlet so that the air from the air inlet uniformly flows to the air outlet through the fan, and an emission head assembly of the plasma generator is arranged close to the air outlet relative to the fan; or
the sterilization and disinfection lamp further comprises a fan, the fan is a reverse centrifugal fan, the air outlet is arranged on the bottom surface of the housing, the air inlet is arranged on the side surface of the housing and/or the air inlet is arranged on the bottom surface of the housing, the fan is used for blowing the positive ions and the negative ions into the outside environment from the air outlet, and an emission head assembly of the plasma generator is arranged close to the air outlet relative to the fan.

9. The sterilization and disinfection lamp according to claim 1, wherein a plurality of air guide ribs are disposed on a surface of the bottom surface of the housing towards the interior of the housing;
the plurality of air guide ribs are arranged in a ring structure, two adjacent air guide ribs are arranged at an interval, an air guide duct is formed between two adjacent air guide ribs, the air passing area of the air guide duct gradually increases along an air outlet direction, one end of the air guide duct communicates with the air inlet, and the other end of the air guide duct communicates with the air outlet; and/or
the air guide rib is an arc-shaped section and the air guide rib is provided with a first radian, and the first radian is greater than 0° and less than 90°.

10. The sterilization and disinfection lamp according to claim 9, wherein the plasma generator comprises:
a plasma flyback transformer, which is mounted on the housing; and
an emission head assembly, which is arranged on a cavity wall of the sterilization cavity, the plasma flyback transformer is connected to the emission head assembly, at least an emission head of the emission head assembly is arranged inside the air guide duct, and at least one emission head assembly is arranged inside at least one air guide duct.

11. The sterilization and disinfection lamp according to claim 10, wherein a plurality of emission head assemblies are provided;
the plurality of emission head assemblies are arranged at intervals along a circumferential direction of the cavity wall of the sterilization cavity, and the emission head of each emission head assembly is exposed in the sterilization cavity; and/or
a distance between two adjacent emission head assemblies in the plurality of emission head assemblies is the same; and/or
the plasma generator comprises one of a high-energy plasma generator and a low-energy plasma generator; and/or
the emission head assembly comprises a positive ion emission head and a negative ion emission head, the positive ion emission head is parallel to the negative ion emission head, and an extension direction of the positive ion emission head and the negative ion emission head is perpendicular or horizontal to an air flow direction in the sterilization cavity.

12. (canceled)

13. The sterilization and disinfection lamp according to claim 1, wherein the air outlet is provided with an air outlet grid, an air guide surface of the air outlet grid is obliquely arranged relative to a horizontal plane, and the air guide surface obliquely extends downward along an air outlet direction.

14. (canceled)

15. (canceled)

16. (canceled)

17. The sterilization and disinfection lamp according to claim 1, wherein the sterilization and disinfection lamp further comprises a radiator;
the radiator is padded between the light-emitting assembly and the bottom surface of the housing; and/or
the radiator is arranged in a cylinder structure, and the air inlet channel of the sterilization and disinfection lamp passes through the radiator.

18. The sterilization and disinfection lamp according to claim 17, wherein the sterilization and disinfection lamp further comprises a fan, and the gas temperature inside the housing may be less than or equal to 50° by arranging the radiator and adjusting the air velocity and air volume of the fan.

19. (canceled)

20. (canceled)

21. (canceled)

22. The sterilization and disinfection lamp according to claim 1, wherein a mounting head of the housing is provided with a first connecting structure, the sterilization and disinfection lamp further comprises a mounting base, the mounting base is provided with a second connecting structure in cooperation with the first connecting structure, and the first connecting structure and the second connecting structure may be connected in place to supply power to the sterilization and disinfection lamp.

23. The sterilization and disinfection lamp according to claim 22, wherein an outer surface of the mounting head is provided with a first mounting thread, the mounting base is provided with a mounting groove, the mounting groove is provided with a second mounting thread in cooperation with the first mounting thread, the mounting base supplies power to the sterilization and disinfection lamp after the first mounting thread and the second mounting thread are connected in place, the first mounting thread serves as the first connecting structure, and the second mounting thread serves as the second connecting structure; or
an outer surface of the mounting head is provided with a first clamping structure, the mounting base is provided with a second clamping structure in cooperation with the first clamping structure, the mounting base supplies power to the sterilization and disinfection lamp after the first clamping structure and the second clamping structure are clamped in place, the first clamping structure serves as the first connecting structure, and the second clamping structure serves as the second connecting structure; or
the first connecting structure is fixedly connected to the second connecting structure; or
the first connection structure is connected to the second connection structure line.

24. The sterilization and disinfection lamp according to claim 22, wherein the sterilization and disinfection lamp further comprises:
a support post, one end of the support post being connected to the mounting base, the support post is a hollow structure, and at least part of wires being accommodated in the support post; and
a chassis, the other end of the support post being connected to the chassis,
wherein the chassis may be mounted on a wall to enable the sterilization and disinfection lamp hang on the wall, or the chassis may be placed on a table top to enable the sterilization and disinfection lamp to stand upright;
the sterilization and disinfection lamp further comprises a cover body, the cover body is connected to the mounting base or the cover body is connected to the support post, and the light-emitting assembly is located in the cover body; and/or
the sterilization and disinfection lamp further comprises a support rod, and the support rod is connected to the chassis and the cover body.

25. (canceled)

26. A combined lamp, comprising:
the sterilization and disinfection lamp according to claim 1; and
an illuminating lamp, and the illuminating lamp is connected to the sterilization and disinfection lamp, the illuminating lamp comprises a lamp housing and a light source arranged inside the lamp housing, and the lamp housing is detachably connected to the housing of the sterilization and disinfection lamp.

27. The combined lamp according to claim 26, wherein the combined lamp comprises at least one light-emitting group, and each light-emitting group is composed of a sterilization and disinfection lamp unit and an illuminating lamp unit, wherein each sterilization and disinfection lamp unit comprises one or a plurality of sterilization and disinfection lamps, each illuminating lamp unit comprises one or a plurality of illuminating lamps, and the light-emitting group comprises:
one sterilization and disinfection lamp unit and one illuminating lamp unit; or
two sterilization and disinfection lamp units and one illuminating lamp unit, and the two sterilization and disinfection lamp units are respectively arranged at both ends of the illuminating lamp unit; or
one sterilization and disinfection lamp unit and two illuminating lamp units, and the two illuminating lamp units are respectively arranged at both ends of the sterilization and disinfection lamp unit.

28. The combined lamp according to claim 26, wherein the combined lamp further comprises a connector and a connecting post, one end of the connecting post is connected to the top surface of the housing of the sterilization and disinfection lamp, the other end of the connecting post is connected to the connector, the illuminating lamp is connected to the sterilization and disinfection lamp or the illuminating lamp is connected to the connecting post or the illuminating lamp is connected to the connector, the connector may be mounted on a wall to enable the sterilization and disinfection lamp hang on the wall, or the connector may be placed on a table top to enable the sterilization and disinfection lamp to stand upright;
the combined lamp further comprises a plurality of connecting rods, one end of the connecting rod is connected to the illuminating lamp, and the other end of the connecting rod is connected to the connector; and
the combined lamp further comprises a lamp ring, the lamp ring is arranged along a circumferential direction of the sterilization and disinfection lamp, the lamp ring is provided with a plurality of lamp mounting positions for mounting the illuminating lamp, and the lamp ring is connected to the connecting rod or the lamp ring is connected to the housing or the lamp ring is connected to the connecting post.

29. (canceled)

30. (canceled)

31. (canceled)

32. (canceled)

33. (canceled)

34. (canceled)
